(19) United States
(12) Patent Application Publication
(10) Pub. No.: US 2011/0272304 A1
(43) Pub. Date: Nov. 10, 2011

(54) Cleaning Wipe for Use With Disinfectants, Method of Manufacture Thereof, and System

(75) Inventors: Sanjay Wahal, Appleton, WI (US); Samuel C. Baer, Atlanta, GA (US); Gary L. Schroeder, Neenah, WI (US)

(73) Assignee: GEORGIA-PACIFIC CONSUMER PRODUCTS LP, Atlanta, GA (US)

(21) Appl. No.: 13/085,091

(22) Filed: Apr. 12, 2011

Related U.S. Application Data
(60) Provisional application No. 61/323,153, filed on Apr. 12, 2010.

(51) Int. Cl. A01N 25/34; B32B 37/14; B32B 37/02; A01P 1/00; B65D 71/00

(52) U.S. Cl. 206/223; 424/443; 156/296

(57) ABSTRACT

A cleaning wipe includes a dry substrate comprising nonwoven synthetic fibers, wherein the fibers have a fineness of about 2.3 denier to about 3.3 denier; and a nonionic surfactant disposed on the dry substrate, wherein the surfactant is present on the dry substrate at an add-on level of about 0.1 weight percent to about 1.5 weight percent, based on the weight of the dry substrate, and wherein the cleaning wipe is active disinfectant stable.

### CROSS-REFERENCE TO RELATED APPLICATION

This application claims priority to U.S. Provisional Application No. 61/323,153 filed Apr. 12, 2010, the disclosure of which is incorporated herein in its entirety by this reference.

### BACKGROUND OF THE INVENTION

The present disclosure generally relates to a cleaning wipe for use with disinfectants and, more particularly, to a dry cleaning wipe that can be used with common disinfectants without appreciably decreasing the efficacy of the disinfectant.

Disinfectants are commonly used on cleaning surfaces to kill micro-organisms and reduce the possibility for infections. Generally, disinfectants can be mixed in a solution and applied to surfaces by either saturating the surface directly with the solution or using a wipe, towel, sponge, or other substrate that is soaked with the disinfectant.

In the field of disinfectants, guidelines exist for the minimum concentration of disinfectant in a disinfectant solution to avoid outbreaks of harmful bacteria and other organisms. The two most common disinfectants in disinfectant solutions are quaternary ammonium chloride-based (commonly referred to as “quats”) or chlorine-based disinfectants. Quats and chlorine are also commonly used as the active ingredient in sanitizers. By definition, “sanitizers” use a lower concentration of quat compounds than are used in “disinfectant” solutions. Typically, a sanitizer will only have 200-400 parts per million (ppm) of a quat or 100 ppm of hypochlorite ion in solution while a disinfectant will have about 600 5000 ppm of a quat or hypochlorite in solution. As such, sanitizers are safe for cleaning surfaces used in food preparation (e.g., restaurants and kitchens), while disinfectants are generally used to clean surfaces in hospitals and other like environments.

A dry wipe can be wetted with the disinfectant solution by the user or it can be pre-saturated by the manufacturer. For the wipe to be effective, the disinfecting solution must maintain a certain concentration of disinfectant. A common problem, however, is that a wipe may deplete about 10-60 percent (%) of the disinfectant (e.g., quat) from the disinfectant solution, depending on the materials making up the construction of the wipe. The woven or nonwoven fabric of the substrate can reduce the concentration of disinfectant in the solution. For example, a nonwoven fabric can be repeatedly rinsed in a disinfectant solution contained in a bucket, while disinfecting surfaces in a hospital. If the nonwoven fabric is diluting or reducing the effectiveness of the disinfectant in the disinfecting solution, then the surfaces are not being disinfected. The same type of problem is also encountered with sanitizes solutions.

Pre-saturated wipes solve, or at least reduce this problem by compensating the disinfectant concentrations in the disinfectant solution during the manufacturing process to be consistent with the desired percentage of active disinfectant in the substrate. In other words, the disinfectant concentration can be increased to account for the depletion of the disinfectant from adsorption by the substrate, and to ensure the desired overall concentration in the wipe. As used herein, the term “pre-saturated” in reference to a wipe, refers to wipes that are saturated by the manufacturer with the desired liquid and delivered to the user in a wet format. However, for products that are delivered to the customer as a dry substrate to which the customer adds their own disinfectant solution, the level of disinfectant in disinfectant solutions cannot be increased. In such instances, the customer must rely on the substrate to release 100% of the disinfectant from the substrate after the solution has been added thereto.

Attempts have been made that address the problem of decreasing disinfectant effectiveness, such as for quat solutions, but these attempts often are not suitable for other disinfectant solutions, such as chlorine-based solutions. In the same way as quat solutions, the active disinfectant of chlorine solutions also adsorbs to untreated wipe substrates. However, active chlorine, as an oxidizer, can also react with wiper substrates or additives. This presents additional constraints for the design of a wiper product for use with active chlorine sanitizes or disinfectant solutions. Again, this is problematic for many end users due to the frequent use of chlorine solutions to disinfect or sanitize a surface. Even those who use quat solutions in some circumstances will often use chlorine solutions in other circumstances. It would be convenient to use the same wiper product for all circumstances.

Accordingly, there remains a need for an improved cleaning wipe that can be used with common disinfectants solutions without appreciably decreasing the efficacy of the active disinfectant therein.

### BRIEF DESCRIPTION OF THE INVENTION

Disclosed herein are cleaning wipes that are stable and compatible for use with both quat-based disinfectant solutions and chlorine-based disinfectant solutions. In one embodiment, the cleaning wipe comprises a thy substrate comprising nonwoven synthetic fibers, wherein the fibers have a fineness of about 2.3 denier to about 3.0 denier; and a nonionic surfactant disposed on the dry substrate, herein the surfactant is present on the dry substrate at an add-on level of about 0.1 weight percent to about 1.5 weight percent, based on the weight of the dry substrate, and wherein the cleaning wipe is active disinfectant stable.

In another embodiment, a cleaning wipe system comprises a cleaning wipe comprising a dry substrate of nonwoven synthetic fibers, wherein the fibers have a fineness of about 2.3 denier to about 3.3 denier; and a nonionic surfactant disposed on the dry substrate, wherein the surfactant is present on the dry substrate at an add-on level of about 0.1 weight percent to about 1.5 weight percent, based on the weight of the dry substrate, and wherein the cleaning wipe is both quat-based disinfectant stable and chlorine-based disinfectant stable; a disinfectant solution; and a container configured to contain the cleaning wipe and the disinfectant solution.

In another embodiment, a method of making a cleaning wipe comprises spunbonding a dry substrate comprising nonwoven polypropylene fibers; and applying a nonionic surfactant to the dry substrate to make the cleaning wipe both quat-based disinfectant stable and chlorine-based disinfectant stable, wherein the surfactant is present on the dry substrate at an add-on level of about 0.1 weight percent to about 1.5 weight percent, based on the weight of the dry substrate.

The above described and other features are exemplified by the following detailed description.

### DETAILED DESCRIPTION OF THE INVENTION

Disclosed herein are cleaning wipes that are stable and compatible for use with disinfectants, and more particularly, for use with both quat-based disinfectant solutions and chlorine-based disinfectant solutions. In one embodiment, a cleaning wipe includes a dry nonwoven substrate comprising synthetic fibers having a fineness of about 2.3 to about 3.3 denier; and a nonionic surfactant disposed on the dry substrate present at an add-on level of about 0.1 weight percent (wt %) to about 1.5 wt % based on the weight of the dry substrate, wherein the cleaning wipe is active disinfectant stable.

The cleaning wipe as described herein can be used with common disinfectants, such as quaternary ammonium chloride (“quat”) solutions or sodium hypochlorite bleach (“chlorine”) solutions, without appreciably decreasing the efficacy of the active disinfectant of the solution under typical usage and storage conditions, particularly in health care and food service institutional settings. The cleaning wipe is considered to be stable with such common disinfectant solutions. Specifically, the addition of a nonionic surfactant, such as an ethoxylated fatty alcohol, into the cleaning wipe prevents the quat solution from being adsorbed on the fibers of the cleaning wipe. The nonionic surfactant provides a wettable substrate fiber, while preventing the fibers from adsorbing (i.e., depleting) the quaternary ammonium chloride from the solution over a period of time.

With regard to chlorine solutions, the synthetic fibers having a fineness of 2.3 to 3.3 denier of the substrate serve to effectively keep the nonionic surfactant on the fiber surfaces and slow the surfactant from migrating into the active chlorine solution where it can react with the active chlorine. Because of this, the oxidation reaction of active chlorine with the nonionic surfactant proceeds much more slowly than cleaning wipes using other fibers. The synthetic fibers of the cleaning wipe described herein, therefore, can advantageously be used with chlorine solutions without appreciably decreasing the efficacy of the active chlorine due to reaction with the surfactant. As used herein, the term “stable” in reference to the use of the cleaning wipe with disinfectant solutions, refers to a cleaning wipe that maintains at least about 85 wt %, specifically about 90 wt %, and more specifically about 95 wt % of an initial active disinfectant concentration after exposure of the disinfectant solution to the dry substrate. Expressed in another manner, the cleaning wipe described herein depletes equal to or less than about 10 wt % of an active disinfectant, specifically equal to or less than about 7.5 wt % active disinfectant, and more specifically equal to or less than about 5 wt % active disinfectant that is introduced in solution to the cleaning wipe, based on the total weight of the active disinfectant. A further advantage is the cleaning wipe described herein remains stable over a period of time that such wipes would be expected to be exposed to such disinfectant solutions (e.g., the time a roll of such wipes would be sitting in a bucket with the disinfectant solution). In one embodiment, the cleaning wipes remain stable for a period of 8 to 24 hours in an institutional setting.

The nonionic surfactants described herein are selected to adsorb or otherwise bond to the fibers of a thy substrate of the cleaning wipe, thereby preventing the active disinfectants from being adsorbed by the fibers of the dry substrate. Without being bound by theory, it is believed that the nonionic surfactants described herein alter the relative equilibrium at the cleaning wipe surface by both modifying the surface to make it less hydrophobic and modifying the disinfectant solution to make it less hydrophilic. For example, in the case of a quat solution, such as a dialkyl or alkyl benzyl quat solution, the net result is a reduced attraction of the hydrophobic wiper surface for the hydrophobic hydrocarbon tails of the quat solution. Nonionic surfactants are a class of materials broadly characterized as being made of molecules containing hydrophilic groups adequately separated from hydrophobic groups. The hydrophobic groups have an affinity for the fiber surface of the substrate. Unlike anionic surfactants, the nonionic nature of the surfactant does not attract the cationic quat-based or chlorine-based disinfectant solutions and prevents the active disinfectant from bonding to the substrate fibers.

The solubility of the nonionic surfactant is one factor in its ability to provide stability to the disinfectant solutions, thereby not appreciably decreasing the efficacy of the active disinfectant. The water solubility of a nonionic surfactant can be predicted by HLB value of the surfactant. “HLB” stands for Hydrophile/Lipophile Balance and is the relationship between the hydrophilic portion of the nonionic surfactant to the lipophilic portion. In other words, HLB represents the ratio of the water-loving portion of the nonionic surfactant to the oil-loving portion of the nonionic surfactant. The lower the HLB value, the more lipophilic or oil soluble the surfactant, the higher the HLB value, the more hydrophilic or water soluble that surfactant. The balance is measured based on the molecular weight of the nonionic surfactant. The HLB value is the molecular weight percent of the hydrophilic portion of the nonionic surfactant, divided by five. Exemplary nonionic surfactants for the cleaning wipes described herein have an HLB value of about 10 to about 20; specifically about 10 to about 18.

The nonionic surfactants utilized herein include those commercially well known and can be, for example, primary aliphatic alcohol ethoxylates, secondary aliphatic alcohol ethoxylates, alkylphenol ethoxylates and ethylene-oxide-propylene oxide condensates with primary alkanols, and condensates of ethylene oxide with sorbitan fatty acid esters. The primary and secondary alcohols can have from about 8 to about 32 or more carbon atoms, and the alkyl groups of the alkylphenols can have from about 6 to about 18 or more carbon atoms. Thus, the nonionic surfactants can generally comprise the condensation products of an organic aliphatic or alkyl aromatic hydrophobic compound and hydrophilic ethylene oxide groups. The hydrophobic compounds can have, for example, a carboxy, hydroxy, amido, or amino group with a free hydrogen attached to the nitrogen that can be condensed with ethylene oxide. Further, the length of the polyethylene glycol chain can be adjusted to achieve the desired balance between the hydrophobic and hydrophilic elements. A mixture of ethylene and propylene groups can also be used to achieve the desired balance between the hydrophobic and hydrophilic elements. In one embodiment, a block copolymer comprising a combination of ethylene oxide blocks and propylene oxide blocks (a polyoxyethylene-polyoxypropylene block copolymer) can be used.

Exemplary nonionic surfactants for the cleaning wipes described herein can comprise water soluble alcohol ethylene oxide condensates of a secondary aliphatic alcohol containing from 9 to 18 carbon atoms in a straight or branched configuration, condensed with from about 5 to 40 moles, specifically from about 7 to 20 moles, of ethylene oxide. Exemplary commercially available nonionic surfactants of this composition are C₁₁-C₁₅ secondary alkanols condensed with 7, 9, 12, 20 or 40 moles of ethylene oxide (alkyloxypolyethylene oxyethanols), produced by Union Carbide under the tradenames Tergitol® 15-S-7, 15-S-9, 15-S-12, 15-S-20, and 15-S-40. Additional exemplary nonionic surfactants, of the same type, are marketed by Union Carbide under the tradenames Tergitol® TMN-6 and TMN-10, believed to comprise reaction products of trimethyl-nonanol with ethylene oxide. Other exemplary nonionic surfactants are commercially available from Ciba under the tradename Irgasurf® HL 560. Still other nonionic surfactants include block copolymers of polyoxyethylene and polyoxypropylene that are available under the trade name Pluronic®, marketed by BASF. A single member of any f the foregoing nonionic surfactant compositions can be used in the cleaning wipe, or mixtures of such exemplary nonionic surfactant materials can be employed.

The nonionic surfactant, e.g., ethoxylated fatty alcohol, will be applied to the dry substrate at an add-on level of less than about 2.5 wt % per weight of the substrate. In an exemplary embodiment, specifically about 0.1 wt % to about 1.5 wt %, and more specifically about 0.6 wt % to about 1.3 wt % of the nonionic surfactant is present in the cleaning wipe, based on the dry weight of the nonwoven dry substrate.

The nonionic surfactant can be applied to the dry substrate by any method effective in bonding the surfactant to the fibers of the substrate, and will depend, at least in part, on the type of surfactant chosen for the cleaning wipe. The nonionic surfactant may be added to fibers prior to conversion into substrates or it may be incorporated into the fiber during melt-extrusion of the fibers. Similarly, the nonionic surfactant may be added to the cleaning wipe substrate at any point during the production of the substrate web. In one embodiment, the nonionic surfactant can be topically applied to the nonwoven substrate after the web has passed over the heated calendar roll bonder and before the web is wound up into a finished roll. The nonionic surfactant may be applied by any of well-known processes that include, without limitation, spray application, gravure printing, brush, foam, slot dye, dip-and-squeeze, saturation, or other similar processes.

Optionally, the cleaning wipes may also incorporate other optional compounds in addition to the nonionic surfactant. Additional optional compounds can include any compounds that enhance the functionality or aesthetics of the cleaning wipe. For example, such optional compounds may include, without limitation, pH buffers, chelating agents, anti-microbial agents, pigments, color stabilizers, softeners, fragrances, and the like.

The nonwoven dry substrate of the cleaning wipe may comprise any suitable matrix of fibers or filaments that are typically consolidated into a nonwoven web. As used herein the term “nonwoven” means a web having a structure of individual fibers or threads which are interlaid, but not in an identifiable manner as in a knitted fabric. Nonwoven substrates have been formed from many processes such as for example, meltblowing, spunbonding, bonded carded web, air laying, wet laying, solution spinning, pattern-roll bonding, through-air bonding, hydro entangling, and other like processes. Staple length fibers, continuous filaments, or blends of fibers and/or filaments having the same or different compositions may be used to form the substrate. Staple lengths are selected in the range of about 0.50 inch to about 3 inches, specifically about 1 to about 2 inches. The fiber denier can be selected in the range of about 1 to about 10 denier per filament (dpf), specifically about 1.2 to about 6 dpf, and more specifically about 2.3 to about 3.3 dpf. Denier is a unit used to indicate the fineness of a filament given by the weight in gramps for 9,000 meters of filament. A filament of 1 denier has a mass of 1 gram for 9,000 meters of length. The diameter of the fibers are selected to be greater than about 5 micrometers, specifically about 5 to about 50 micrometers; and more specifically about 19 to about 30 micrometers.

The fibers and/or filaments may be selected from natural or synthetic composition and they may be homogeneous or mixed fiber/filament length. Synthetic fibers, which may be blended in whole or part, include, but are not limited to, thermoplastic and thermoset polymers. In applications where the user is expected to add the disinfecting solutions to the wipe substrate at time of use, the exemplary wipe substrate composition will comprise a majority of synthetic fibers, specifically one-hundred percent synthetic fibers. Moreover, in an exemplary embodiment where chlorine solutions will be the disinfectant of choice for the cleaning wipes, the substrate comprises polypropylene fibers; specifically spunbond polypropylene fibers; and more specifically spunbond polypropylene having a fineness of about 2.3 to about 3.3 denier. As mentioned above, it has been unexpectedly found that synthetic fibers, and particularly polypropylene fibers in the cleaning wipe substrate help to slow the loss of active chlorine in the disinfectant solution from reaction with the oxidizing species of the nonionic surfactant. The polypropylene fibers serve to effectively keep a majority of the nonionic surfactant on the fiber surfaces rather than permitting the surfactant to migrate into the chlorine disinfectant solution where it can react with the active chlorine and diminish the efficacy of the wipe.

Thermoplastic polymers for use in the nonwoven dry substrate can include, without limitation, polyolefins, polyamides and polyesters. The thermoplastic polymers may be further selected from homopolymers, copolymers, conjugates and other derivatives including those thermoplastic polymers having incorporated melt additives or surface-active agents. Exemplary thermoplastic fibers can include, without limitation, polyesters, nylons, polypropylenes, polyethylenes, acrylics, polyvinyls, polyurethanes, and other such synthetic fibers as are well known. Exemplary polyolefins include, but are not limited to, polyethylene, polypropylene, polybutylene, and the like; exemplary polyamides include, but are not limited to, nylon 6, nylon 6/6, nylon 10, nylon 12 and the like; and exemplary polyesters include, but are not limited to, polyethylene terephthalate, polybutylene terephthalate and the like. The nonwoven dry substrate may additionally have more than one type of fiber, may have biconstituent fibers, or may have conjugate fibers.

The cleaning wipes described herein can be made of nonwoven substrate webs that are a single layer web or multiple layers. A substrate web made of multiple layers may have similar materials in each layer or may be made of differing layers. The cleaning wipe may also be a multilayer laminate.

In an exemplary embodiment, the nonwoven dry substrate comprises spunbond filaments, specifically polypropylene spunbond filaments. As used herein, the term “spunbond” and “spunbond filaments” refers to continuous filaments which are formed by extruding a molten thermoplastic material (e.g., polypropylene) as filaments from a plurality of fine, usually circular, capillaries of a spinnerette with the diameter of the extruded filaments then being rapidly reduced as by, for example, eductive drawing and/or other well-known spun-bonding mechanisms. Spunbond fibers can include monocomponent, multicomponent, and/or biconstituent fibers. In addition, although spunbond filaments are typically round, filaments having various geometric or irregular shapes can also be used in connection with the nonwoven dry substrate. Other spunbond webs can comprise polyamide (e.g., nylon), polyester, or other like polymers.

It has also been discovered, that apart from substantially preventing the depletion of disinfectant from a solution, the cleaning wipes as described herein also release more of the disinfectant fluid compared to other nonwoven cleaning wipes. The nonwoven webs comprised of fine filaments (such as meltblown fibers) have fine capillary pores that lock the disinfectant solution more tightly into the substrate due to higher capillary pressures resulting from the smaller pores. As such, the fine diameter meltblown substrates do not release as much fluid during wiping as a spunbond substrate made of thicker fibers or filaments as described herein. For example, a dry substrate made of spunbond polypropylene as described above readily releases more disinfectant solution compared to meltblown and other nonwovens of finer diameter. Moreover, because the substrate described herein readily releases more fluid, a longer wipe-dry exists for the cleaning wipe over the same wiping time and solution loading level when compared to other nonwoven substrates. The longer wipe-dry can result in killing more bacteria and making the cleaning wipe overall more effective for disinfection. As used herein, the term “wipe-dry” is intended to generally refer to the time for which the cleaning wipe can release fluid (i.e., leave a film or puddle of solution on a surface) before the surface being wiped becomes dry. An example of this longer wipe-thy is shown in the Example section below.

It is intended that the nonwoven substrate described herein be substantially dry and the resulting cleaning wipe be substantially dry when delivered to the user. As used herein, the term “substantially dry” refers to the substrate being free of liquid and all but ambient moisture. The cleaning wipes can be delivered, for example, in a stack of the nonwoven dry substrates. As used herein, the term “stack” is used broadly to include any collection of the cleaning wipes wherein there is a plurality of surface-to-surface interfaces of the thy substrates. This not only includes a vertically stacked collection of individual wipes, but also includes a horizontally stacked collection of individual wipes, as well as a rolled or folded collection of continuous cleaning wipe material.

The stacked cleaning wipes can be stored in a sealable container such as, for example, within a bucket with an attachable lid, sealable plastic pouches or bags, canisters, jars, tubs, and the like. In an exemplary embodiment, the cleaning wipe stack is maintained in a resealable container. A resealable container can be useful in reducing the evaporation of solution from the wipes. A selected amount of disinfectant solution can then be added to the container such that the dry nonwoven substrates of the cleaning wipes contain the desired amount of disinfectant. In one embodiment, the stacked cleaning wipes are placed or formed in the container and the disinfectant solution added thereto. The amount and composition of the disinfectant solution added to the dry substrates will vary with the desired application and/or function of the wipes. In an exemplary embodiment, the cleaning wipes are saturated and/or moistened with the disinfectant solution and the wipes are capable of substantially uniformly retaining the disinfectant solution over extended periods of time. The cleaning wipes as described herein have an aqueous fluid absorbency of about 5 to about 10 grams (fluid) per gram (wipe) based on the standard basket absorbency test. This is particularly advantageous in that cleaning wipes taken from the top of the stack will contain substantially the same amount of disinfectant solution as those taken later and/or from the bottom of the stack. Moreover, the nonionic surfactant in the nonwoven substrates substantially prevents the depletion of quat disinfectant from the solution contained in the wipe. The cleaning wipe can subsequently be used to wipe a surface and/or act as a vehicle to deliver and apply disinfectant to a surface. The saturated and/or moistened cleaning wipe can be used to treat various surfaces. As used herein, “treating” surfaces is used in the broad sense to include, without limitation, disinfecting, sanitizing, cleaning, washing, and the like. The cleaning wipes are well suited to treat surfaces such as, without limitation, counters, tables, furniture, workstations, windows, lab tops, equipment, machinery, floors, walls, and the like.

Embodiments of the cleaning wipe are provided in the examples below, however the following examples are not meant to limit the scope of the present invention. The examples illustrate a nonwoven substrate including an nonionic surfactant. With the nonionic surfactant formulation the dry nonwoven substrate does not readily deplete the disinfectant concentration in the disinfectant solution.

### EXAMPLES

In Examples 1 and 2, spunbond polypropylene substrates were wetted with a disinfectant solution at 6.0 grams solution per gram of dry substrate wipe. After the desired contact time between the substrate and the disinfectant solution, the active disinfectant concentrations were determined for each solution.

### Example 1

The first example included two spunbond polypropylene wipe samples; Wipe 1 containing 0.7 wt % Tergitol 15-S-70 surfactant; and Wipe 2 containing 1.2 wt % Tergitol 15-S-7® surfactant. To achieve the desired Tergitol surfactant add-on, a controlled amount of aqueous solution of Tergitol 15-S-7 is sprayed and allowed to dry on the polypropylene spunbond web during the manufacturing process. In order to calculate/verify the weight percentage of the Tergitol surfactant in the wipe, a portion of each sample (about 5 grams each) was weighed and extracted with methanol using a four-hour Soxhlet extraction. The extracts were collected in weighed Soxhlet beakers and evaporated to dryness using low heat. The beakers were heated an additional 30 minutes at 70 degrees Celsius, cooled in a desiccator to room temperature, and weighed again. Weight percent extract was calculated from this data. The extract for Wipe 2 was analyzed by nuclear magnetic resonance (NMR) spectrometry to determine the fraction of Tergitol surfactant in the extract. This was multiplied times the weight percent extract to calculate weight percent Tergitol 15-S-7 surfactant. For Wipe 1, the weight percent extract from untreated fibers (the blank) was assumed to be the same value as for Wipe 2 (since the same spunbond polypropylene was used for both Wipes). This value was subtracted from the weight percent extract for Wipe 1 to calculate the weight percent Tergitol 15-S-7 surfactant in Wipe 1.

A 3 gram section of each wipe (1 and 2) was cut into 2-inch squares and placed into a 3-inch by 3-inch by 1.8 inch high polypropylene tray (with removable, sealable lid). An 18.0 gram aliquot of a 608 ppm (0.0608%) KayQuat II® disinfectant solution was added to the wiper material in each sample tray and the lid immediately sealed onto the tray. KayQuat II® is a quaternary ammonium disinfectant composition commercially available from Kay Chemical Company. The completed Wipe 1 and Wipe 2 samples were tested after 1 hour exposure to the disinfectant solution. This preparation cycle was then repeated three more times for additional durations of 1 day, 3 days, and 7 days.

At the end of the desired time, the pieces for each of Wipe 1 and Wipe 2 were placed into a 12 milliliter, 0.45 micrometer glass microfiber (GMF) Autovial® filter. A plunger was depressed to express the disinfectant solution into a 20 milliliter polypropylene vial. All pieces for the same wipe were processed using the same filter and collected in the same 20 milliliter vial. Each sample solution was diluted to 10 milliliter to 25 milliliter with 5 mM methanesulfonic acid in 40/60 acetonitrile/water, filtered (same type of filters), and transferred to a 1.5 milliliter polypropylene autosampler vial. These filtered solutions were analyzed by liquid chromatography using 262 nanometer ultraviolet absorbance detection (method KayQuat II). Quantitation was based on peak area of the benzalkonium chloride peaks versus external standards in the same eluent.

Tables 1 and 2 illustrate the results of the quat-based disinfectant concentration after the various exposure durations. Table 1 contains the solution concentration of the quat-based active disinfectant after listed contact time on the spunbond polypropylene wipes. Table 2 contains the percent loss of quat-based active disinfectant by adsorption on the spundbond polypropylene wipes (as calculated from the values of Table 1).

TABLE 1

| | Tergitol | Initial | Conc. | Conc. | Conc. | Conc. | Average |
|---|---|---|---|---|---|---|---|
| Wipe | 15-S-7 | Conc. | (ppm) at | (ppm) at | (ppm) at | (ppm) at | Conc. |
| Number | (wt %) | (ppm) | 1 Hour | 1 Day | 3 Days | 7 Days | (ppm) |
| 1 | 0.7 | 608 | 567 | 573 | 572 | 576 | 572 |
| 2 | 1.2 | 608 | 573 | 575 | 581 | 582 | 578 |

TABLE 2

| | | Wt % Loss | Wt % Loss | Wt % Loss | Wt % Loss | Average |
|---|---|---|---|---|---|---|
| | Tergitol | Active | Active | Active | Active | Wt % Loss |
| Wipe | 15-S-7 | Quats at 1 | Quats at 1 | Quats at 3 | Quats at 7 | Active |
| Number | (wt %) | Hour | Day | Days | Days | Quats |
| 1 | 0.7 | 6.7 | 5.8 | 5.9 | 5.3 | 5.9 |
| 2 | 1.2 | 5.8 | 5.4 | 4.4 | 4.3 | 5.0 |

As seen from the tables, the spunbond polypropylene with 1.2 wt % Tergitol 15-S-7 surfactant (Wipe 2) adsorbed less active KayQuat II disinfectant than did Wipe 1 (spunbond polypropylene with 0.7 wt % Tergitol 15-S-7). Therefore, Wipe 2 would appear to be a better choice for a cleaning wipe to be used with a quat-based disinfectant solution.

### Example 2

Spunbond polypropylene cleaning wipes were treated with 0.7 wt % and 1.2 wt % Tergitol 15-S-7® in the same manner as Example 1 to form samples Wipe 3 and Wipe 4, respectively. For each wipe (3 and 4), a 6.00 grain section of the wipe was cut into approximately 2-inch squares and placed into a 3-inch by 3-inch by 1.8-inch high polypropylene tray (with removeable, sealable lid). A 36.0 gram aliquot of a 200 milligram/liter (0.020%) active chlorine bleach disinfectant solution was added to the wiper material in each sample tray and the lid immediately sealed onto the tray.

At 48 hours (2 days) after sample preparation, the sample container was opened and approximately the top half of the wipe stack was lifted slightly. About half a test strip (pHydrion® Micro Chlorine test strips commercially available from Micro Essential Laboratories) was placed between the two half-stacks of wetted cleaning wipes while holding the other half of the test strip. The top wipe half-stack was dropped onto the text strip and pressed down slightly so that the test strip was wetted with the disinfectant solution The top wipe half-stack was then lifted and the test strip removed. The test strip was immediately blotted dry with a paper towel and compared to the reference color chart to determine the concentration of active chlorine bleach. The test strips used in this example had a detection limit of 10 milligrams per liter active chlorine.

Table 3 shows the active chlorine bleach remaining in the wipe samples containing the listed level of Tergitol 15-S-70 surfactant after 48 hours with 6 grams of 200 milligram per liter (mg/L) active chlorine bleach per gram of dry wipe weight.

TABLE 3

| | | Active Chlorine Bleach |
|---|---|---|
| Wiper Number | Tergitol 15-S-7 (Wt %) | (mg/L) after 48 hours |
| 3 | 0.7 | 150 |
| 4 | 1.2 | 150 |

As seen in the table, each wipe reduced the active chlorine bleach level from 200 mg/L to about 150 mg/L. However, it is likely that when used with bleach solutions having higher concentrations of active chlorine levels, the reduction of active chlorine will be a much smaller percentage of the initial active chlorine level. Therefore, from the data of Table 3, either Wipe 3 or Wipe 4 would likely be acceptable for use with chlorine-based disinfectant solutions. That being said, while Wipe 4 was quickly wetted by the bleach solution, Wipe 3 needed about 10 seconds for the bleach solution to wet the wipes. Sample 3, therefore, may not be wetted properly when a bleach solution is poured onto a stack as, for example, oriented in a bucket of stacked wipes.

In summary, spunbond polypropylene wipes with 1.2 wt % Tergitol 15-S-7® surfactant provided quick wettability, low adsorption of KayQuat HO disinfectant, and limited reduction of active chlorine bleach.

### Example 3

Wound rolls of spunbond polypropylene wipes produced by Atex® Corp. containing 0.97 wt % Tergitol 15-S-7® were each wetted with 0.500 gallons (1.89 liters) of a test active chlorine disinfectant solution. Wound rolls of meltblown Kimtech Prep® WetTask® 06411 were also wetted with 0.500 gallons (1.89 liters) of the test active chlorine disinfectant solution. After the desired contact times, the concentration of active chlorine remaining was determined for each solution.

A completed roll of each cleaning wipe was placed into a polyethylene tub. A 0.500-gallon (1893-mL) volume of a diluted bleach disinfectant solution was added to the wiper material in each tub. The tub was immediately sealed with a lid. For the Atex® wiper, duplicate samples were prepared for each listed concentration of active chlorine disinfectant. For the Kimtech Prep® wiper, duplicate samples were prepared for each listed concentration of KayQuat II® disinfectant.

At the end of the desired time, a known volume of sample liquid was removed from the liquid pool at the bottom of a sample tub by pipet and titrated using the method summarized below. The samples were taken from the liquid at the bottom of each sample tub rather than squeezing liquid from wipers in order to minimize loss of active chlorine due to increased evaporation during removal and squeezing individual wipers.

The following solutions were added to a clean disposable 250-mL polystyrene beaker.
1. 10.0 mL deionized water for 500 mg/L target samples, or 26.0 mL deionized water for 3000 mg/L target samples, or 28.0 mL deionized water for 5500 mg/L target samples
2. 1.0 mL 0.50 M Potasium Iodine in deionized water
3. 5.0 mL 1.00 N sulfuric acid in deionized water
4. 0.50 mL 1.2% (12 g/L) (NH₄)₂MnO₄.4H₂O in deionized water
5. 0.50 mL Starch Solution (Fischer Scientific catalog number SS408-1)
6. 20.0 mL of the sample pulled from the tub for 500 mg/L target samples, or 4.0 mL sample for 3000 mg/L target samples, or 2.0 mL sample for 5500 mg/L target samples
7. 10 mL deionized water (rinsing the beaker sides as this is added)

The contents of the beaker were then titrated with 0.0500 N sodium thiosulfate using a Methrohm® Dosimat 665™ titrator. When the endpoint was near (light blue or bluish-brown), an additional 0.50 mL Starch Solution was added. The titration to a clear solution was then completed. The volume of titrant was recorded for each sample.

The mg/L (ppm) active chlorine was calculated from this data, using 35,453 grains active chlorine per equivalent to complete the calculations. The percent loss of active chlorine was then calculated from this data and is shown in Table 4.

TABLE 4

| | Initial Conc. | Conc. | % Loss | Conc. | % Loss | Conc. | % Loss |
|---|---|---|---|---|---|---|---|
| Wiper | (ppm) Active | (ppm) at | at 1 | (ppm) at | at 2 | (ppm) at | at 3 |
| MadeBy | Chlorine | 1 Day | Day | 2 Days | Days | 3 Days | Days |
| Atex ® | 485 | 464 | 4.3 | 393 | 19.0 | 320 | 34.0 |
| Atex ® | 3001 | 2848 | 5.1 | 2336 | 22.2 | 1899 | 36.7 |
| Atex ® | 5543 | 5340 | 3.7 | 4942 | 10.8 | 4465 | 19.4 |
| KC | 485 | 432 | 10.9 | 372 | 23.3 | 305 | 37.1 |
| KC | 3001 | 2953 | 1.6 | 2834 | 5.6 | 2779 | 7.4 |
| KC | 5543 | 5427 | 2.1 | 5410 | 2.4 | 5390 | 2.8 |

The Atex® spunbond polypropylene cleaning wipes containing 0.97% Tergitol 15-S-7® surfactant were tested with diluted bleach sanitizes solutions in the range of 500 to 5500 mg/L active chlorine. Table 4 indicates that losses of active chlorine were minimal (5.1% loss or less) up to 24 hours after wetting the wiper rolls, but higher at 48 hours or longer. The active chlorine loss for the Atex® spunbond cleaning wipe samples initially containing 485 mg/L of solution at 2 and 3 days was comparable to the loss experienced by the meltblown fibers of the Kimtech Prep® wipes. However, the Kimtech Prep® wipes lost more than twice the active chlorine in 24 hours than was lost by the spunbond polypropylene fibers of the Atex® cleaning wipe samples. As described above, this lower loss of active chlorine (compared to samples without the spunbond polypropylene fibers) is likely due to the surface of the spunbond polypropylene fibers holding much of the nonionic surfactant on the wiper surface. Therefore, a much lower concentration of surfactant was in solution and available to react with that active chlorine. The cleaning wipes comprising spunbond polypropylene fibers unexpectedly achieve better (i.e., lower) loss of active chlorine at 24 hours or less, particularly for low concentrations of the disinfectant solution, when compared to cleaning wipes of different fibers.

### Example 4

Wipe residue tests were conducted for spundbond, spunlace, and meltblown polypropylene cleaning wipes. A quaternary amine (“quat”) disinfectant solution was added to the wipes and the amount of liquid left behind on a surface after being wiped was measured. The amount of liquid (or residue) left behind were compared to determine which type of wipe released the greatest amount of quat disinfectant solution.

To begin the test, sample specimens of each type of polypropylene cleaning wipe (meltblown, spunlace, and spunbond) were cut to a 6-inch by 7-inch size with the long dimension being in the machine direction of the wipe. Each sample specimen was then weighed and placed in a quart-size re-sealable plastic bag, the weight of which was also measured. A sample specimen was placed in the bag. To calculate the amount of quat disinfectant solution needed for the desired loading, the desired loading was multiplied by the specimen weight. For this experiment, the liquid loading amount was chosen to achieve about 6.5 times or 6.5 grams (fluid) per gram (wipe) of target loading. The bag containing the specimen was then placed on a scale and the quat disinfectant solution was added in small amounts until the calculated weight was obtained. Because some of the liquid solution would remain behind in the plastic bag when the specimen was removed, 0.2 to 0.5 grams extra solution was added to get close to the desired solution loading in the specimen. After adding the quat disinfectant solution to the bag, the bag was re-sealed, placed on a flat surface, and the solution was gently pushed to each corner of the specimen to ensure even wetting. The sheet was then left to sit in the solution for 30 minutes before testing.

In the meantime, the test surface upon which the specimen would be wiped was removed from the custom rub test machine and placed on a balance. The weight of the test surface was tared and the surface was placed back onto the custom rub test machine. The test surface was held on the machine by trips of Velcro® tape. After the 30 minute soak, the specimen was removed from the bag and attached to the rub block of the custom rub test machine with a specimen clamp. The long dimension (7-inch) of the specimen was placed parallel to the direction of the stroke (i.e., wiping action). The rub block was then rotated so the specimen laid flat on the text surface. The custom rub test machine was then started and the specimen was rubbed on the test surface for a total of 5 strokes. A single stroke was considered one back and forth motion of the specimen across the test surface. At the end of the fifth stroke, the rub block was removed from the test surface. The test surface was again removed from the custom rub test machine and placed back on the balance. The weight of the residue on the test surface was then measured in grams. The re-sealable plastic bag from which the specimen was removed was weighed to determine the weight of solution left in the bag. The actual quat disinfectant solution loading for the specimen could then calculated by subtracting the grams of liquid left in the bag from the grams of liquid added to the specimen and then dividing this number by the specimen weight in grams. This test method was repeated for each sample specimen.

A set of twelve sample specimens were tested for each type of cleaning wipe. The average residue weight and standard deviation were calculated for each set of six sample specimens. The spunbond cleaning wipes were spunbond polypropylene wipes commercially available from Polymer Group, Inc. (PGI), and the results of the wipe residue tests are shown below in Table 5.

TABLE 5

| | | Quat | | | Actual | |
|---|---|---|---|---|---|---|
| PGI SBPP | Dry Wt | Added | Bag Wt | Quat Left | Loading | Residue |
| Specimen | (g) | (g) | (g) | in Bag (g) | (g) | Wt. (g) |
| 1 | 1.156 | 7.514 | 5.360 | 1.906 | 4.851 | 0.381 |
| 2 | 1.204 | 7.740 | 5.408 | 2.194 | 4.606 | 0.352 |
| 3 | 1.185 | 8.097 | 5.400 | 28.22 | 4.451 | 0.305 |
| 4 | 1.215 | 6.181 | 5.401 | 1.231 | 4.074 | 0.336 |
| 5 | 1.210 | 6.509 | 5.434 | 1.333 | 4.278 | 0.268 |
| 6 | 1.202 | 6.830 | 5.510 | 1.562 | 4.383 | 0.301 |
| 7 | 1.172 | 8.170 | 5.383 | 2.591 | 4.760 | 0.365 |
| 8 | 1.228 | 7.505 | 5.373 | 2.063 | 4.432 | 0.305 |
| 9 | 1.234 | 7.192 | 5.551 | 1.431 | 4.669 | 0.265 |
| 10 | 1.181 | 7.641 | 5.506 | 1.795 | 4.950 | 0.327 |
| 11 | 1.171 | 7.084 | 5.512 | 1.545 | 4.730 | 0.326 |

| | |
|---|---|
| Average Residue Weight | 0.321 |
| Standard Deviation | 0.037 |

The spunlace cleaning wipes were spunlace polypropylene wipes commercially available from Kimberly-Clark Corporation (KC) under the tradename KIMTECH PREP® WIPER 06211, and the results of the wipe residue tests are shown below in Table 6.

TABLE 6

| | | Quat | | | Actual | |
|---|---|---|---|---|---|---|
| KC 06211 | Dry Wt | Added | Bag Wt | Quat Left | Loading | Residue |
| Specimen | (g) | (g) | (g) | in Bag (g) | (g) | Wt. (g) |
| 1 | 1.168 | 7.587 | 5.396 | 0.921 | 5.707 | 0.280 |
| 2 | 1.144 | 7.393 | 5.390 | 0.805 | 5.759 | 0.222 |
| 3 | 1.108 | 6.343 | 5.383 | .630 | 5.156 | 0.256 |
| 4 | 1.083 | 6.622 | 5.375 | 0.695 | 5.473 | 0.225 |
| 5 | 1.112 | 6.544 | 5.443 | 0.662 | 5.290 | 0.282 |
| 6 | 1.108 | 6.918 | 5.467 | 0.953 | 5.384 | 0.240 |
| 7 | 1.204 | 6.868 | 5.340 | 0.530 | 5.264 | 0.256 |
| 8 | 1.188 | 8.066 | 5.314 | 1.210 | 5.771 | 0.208 |
| 9 | 1.146 | 6.751 | 5.517 | 0.444 | 5.503 | 0.237 |
| 10 | 1.165 | 6.213 | 5.684 | 0.350 | 5.033 | 0.243 |
| 11 | 1.149 | 6.813 | 5.479 | 0.558 | 5.444 | 0.266 |
| 12 | 1.191 | 7.150 | 5.487 | 0.558 | 5.535 | 0.216 |

| | |
|---|---|
| Average Reside Weight | 0.244 |
| Standard Deviation | 0.024 |

The meltblown cleaning wipes were meltblown polypropylene wipes commercially available from Kimberly-Clark Corporation (KC) under the tradename KIMTECH PREP® WIPER 06411, and the results of the wipe residue tests are shown below in Table 7.

TABLE 7

| | | Quat | | | Actual | |
|---|---|---|---|---|---|---|
| KC 06411 | Dry Wt | Added | Bag Wt | Quat Left | Loading | Residue |
| Specimen | (g) | (g) | (g) | in Bag (g) | (g) | Wt. (g) |
| 1 | 0.958 | 6.352 | 5.430 | 0.400 | 6.213 | .0180 |
| 2 | 0.929 | 6.772 | 5.380 | 0.506 | 6.745 | 0.207 |
| 3 | 0.914 | 5.785 | 5.455 | 0.410 | 5.881 | 0.175 |
| 4 | 0.866 | 5.718 | 5.411 | 0.483 | 6.045 | 0.140 |
| 5 | 0.895 | 5.621 | 5.463 | 0.323 | 5.920 | 0.139 |
| 6 | 0.895 | 5.587 | 5.434 | 0.416 | 5.778 | 0.132 |
| 7 | 0.930 | 5.834 | 5.369 | 0.288 | 5.963 | 0.124 |
| 8 | 0.909 | 6.304 | 5.307 | 0.495 | 6.391 | 0.184 |
| 9 | 0.892 | 6.530 | 5.577 | 0.706 | 6.529 | 0.17 |
| 10 | 0.927 | 6.173 | 5.573 | 0.400 | 6.228 | 0.19 |
| 11 | 0.884 | 5.757 | 5.415 | 0.349 | 6.118 | 0.151 |
| 12 | 0.891 | 5.880 | 5.411 | 0.346 | 6.211 | 0.157 |

| | |
|---|---|
| Average Reside Weight | 0.162 |
| Standard Deviation | 0.026 |

The spundbond polypropylene fibers released the greatest amount of disinfectant solution with an average wipe residue of 0.32 grams. This was greater than the amount of solution released by the spunlace polypropylene fibers (0.24 grams) and nearly twice the amount released by the meltblown polypropylene fibers (0.16 grams). As expected, the meltblown fibers released the least amount of disinfectant solution during the wiping, because the small pores in the fine meltblown fibers create higher capillary pressures that hold in the solution much tighter compared to spunbond fibers. These meltblown fibers had an average fiber diameter of about 4 micrometers. While spunlace fibers generally are not as fine as meltblown fibers, they still have smaller pores, and therefore, higher capillary pressures than spunbond fibers. As such, the spunlace fibers released less disinfectant solution during the wiping. The spunlace fibers had an average fiber diameter of about 13 micrometers. The spunbond polypropylene wipe, having the thickest fibers (average fiber diameter of about 22 micrometers), released the most disinfectant solution during the wiping, because the capillary forces between the solution and the fibers is much lower than for the other cleaning wipes.

Ranges disclosed herein are inclusive and combinable (e.g., ranges of “up to about 25 wt %, or, more specifically, about 5 wt % to about 20 wt %”, is inclusive of the endpoints and all intermediate values of the ranges of “about 5 wt % to about 25 wt %,” etc.). “Combination” is inclusive of blends, mixtures, alloys, reaction products, and the like. Furthermore, the terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by context, (e.g., includes the degree of error associated with measurement of the particular quantity). The suffix “(s)” as used herein is intended to include both the singular and the plural of the term that it modifies, thereby including one or more of that term. Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

While the invention has been described with reference to exemplary embodiments, it will be understood by those skilled in the art that various changes may be made and equivalent elements may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed for carrying this invention, but that the invention will include all embodiments falling within the scope of the appended claims.

What is claimed is:

1. A cleaning wipe, comprising:
a dry substrate comprising nonwoven synthetic fibers, wherein the fibers have a fineness of about 2.3 denier to about 3.3 denier; and
a nonionic surfactant disposed on the dry substrate, wherein the surfactant is present on the dry substrate at an add-on level of about 0.1 weight percent to about 1.5 weight percent, based on the weight of the dry substrate, and wherein the cleaning wipe is active disinfectant stable.

2. The cleaning wipe of claim 1, wherein the cleaning wipe is quat-based disinfectant stable and chlorine-based disinfectant stable.

3. The cleaning wipe of claim 1, wherein the nonionic surfactant comprises an ethoxylated fatty alcohol.

4. The cleaning wipe of claim 3, wherein the ethoxylated fatty alcohol surfactant comprises a condensate of a secondary alcohol having from 9 to 18 carbon atoms with from 5 to 30 moles of ethylene oxide.

5. The cleaning wipe of claim 3, wherein the ethoxylated fatty alcohol surfactant is a condensate of C₁₁-C₁₅ secondary alkanols with 7 to 12 moles of ethylene oxide.

6. The cleaning wipe of claim 5, wherein the ethoxylated fatty alcohol surfactant is an alkyloxypolyethylene oxyethanol wherein the alkoxy groups are derived from C₁₁-C₁₅ secondary alkanols.

7. The cleaning wipe of claim 6, wherein the alkyloxypolyethylene oxyethanol is present on the dry substrate at an add-on level of about 0.6 weight percent to about 1.3 weight percent, based on the weight of the dry substrate.

8. The cleaning wipe of claim 1, wherein the dry substrate comprises a spunbond substrate.

9. The cleaning wipe of claim 8, wherein the synthetic fibers comprise polypropylene fibers.

10. The cleaning wipe of claim 9, wherein the polypropylene fibers have a diameter of greater than about 5 micrometers.

11. The cleaning wipe of claim 9, wherein the polypropylene fibers have a diameter of about 19 micrometers to about 30 micrometers.

12. A cleaning wipe system for disinfecting surfaces, comprising:
a cleaning wipe comprising a dry substrate comprising nonwoven synthetic fibers, wherein the fibers have a fineness of about 2.3 denier to about 3.3 denier; and a nonionic surfactant disposed on the dry substrate, wherein the surfactant is present on the dry substrate at an add-on level of about 0.1 weight percent to about 1.5 weight percent, based on the weight of the dry substrate, and wherein the cleaning wipe is both quat-based disinfectant stable and chlorine-based disinfectant stable;
a disinfectant solution; and
a container configured to contain the cleaning wipe and the disinfectant solution.

13. The cleaning wipe system of claim 12, wherein the disinfectant solution is a quaternary ammonium chloride solution or a sodium hypochlorite bleach solution.

14. The cleaning wipe system of claim 12, wherein the nonwoven synthetic fibers comprise spunbond polypropylene fibers having a diameter of about 19 micrometers to about 30 micrometers.

15. The cleaning wipe system of claim 12, wherein the nonionic surfactant comprises an ethoxylated fatty alcohol.

16. The cleaning wipe system of claim 15, wherein the ethoxylated fatty alcohol surfactant comprises a condensate of a secondary alcohol having from 9 to 18 carbon atoms with from 5 to 30 moles of ethylene oxide.

17. The cleaning wipe system of claim 15, wherein the ethoxylated fatty alcohol surfactant is a condensate of C₁₁-C₁₅ secondary alkanols with 7 to 12 moles of ethylene oxide.

18. The cleaning wipe system of claim 17, wherein the ethoxylated fatty alcohol surfactant is alkyloxypolyethylene oxyethanol wherein the alkoxy groups are derived from C₁₁-C₁₅ secondary alkanols.

19. A method of making a cleaning wipe, comprises:
spunbonding a dry substrate comprising nonwoven polypropylene fibers; and
applying a nonionic surfactant to the dry substrate to render the cleaning wipe both quat-based disinfectant stable and chlorine-based disinfectant stable, wherein the surfactant is present on the dry substrate at an add-on level of about 0.1 weight percent to about 1.5 weight percent, based on the weight of the dry substrate.

20. The method of claim 19, wherein the nonionic surfactant is applied after formation of the dry substrate.

21. The method of claim 19, wherein the nonionic surfactant is topically applied to the polypropylene nonwoven web after the web passes a heated calendar roll bonder and before the web is wound into a finished roll.

22. A cleaning wipe produced by the method of claim 19.
